(19) United States
(12) Patent Application Publication
(10) Pub. No.: US 2015/0202136 A1
(43) Pub. Date: Jul. 23, 2015

(54) TOPICAL USE OF A SKIN-COMMENSAL PREBIOTIC AGENT AND COMPOSITIONS CONTAINING THE SAME

(71) Applicant: The Procter & Gamble Company, Cincinnati, OH (US)

(72) Inventors: Anthony Charles Lanzalaco, Fairfield, OH (US); Duane Larry Charbonneau, Mason, OH (US); Brian Wilson Howard, Liberty Township, OH (US)

(21) Appl. No.: 14/378,941

(22) PCT Filed: Feb. 12, 2013
(86) PCT No.: PCT/US13/25736

§ 371 Date: Aug. 14, 2014

Related U.S. Application Data
(60) Provisional application No. 61/598,594, filed on Feb. 14, 2012.

(51) Int. Cl. A61K 8/60; A61Q 19/02; A61Q 19/10; A61Q 17/00; A61Q 19/00; A61Q 19/08

(52) U.S. Cl. CPC A61K 8/60; A61Q 19/008; A61Q 19/007; A61Q 19/08; A61Q 19/10; A61Q 17/00; A61Q 19/02

(57) ABSTRACT

The topical use of a skin commensal prebiotic to improve the health of the skin microbiome, thereby potentially improving the condition and/or appearance of the skin, and topical cosmetic compositions that include the skin commensal prebiotic. The topical cosmetic compositions may include a dermatologically acceptable carrier and an effective amount of prebiotic, and may be used in conjunction with one or more oral or topical prebiotics, probiotics and/or probiotic lysates.

### FIELD OF THE INVENTION

The compositions and methods herein relate generally to the use of a prebiotic agent for skin commensal microorganisms. More specifically, the compositions and methods herein relate to a topically applied prebiotic agent.

### BACKGROUND OF THE INVENTION

The skin and gastrointestinal (“GI”) tracts of most humans are colonized by a diverse array of microorganisms. Colonization generally begins shortly after birth when an infant is exposed to the maternal microflora and other environmental events that typically lead to the colonization of a previously, gnotobiotic human fetus. From the time of initial colonization, the human microbiome remains in a state of flux where the composition of the resident microflora changes over time in response to factors intrinsic and extrinsic to the host. In general, the microorganisms that colonize human hosts may be grouped into three distinct categories: (1) those that are sporadic residents and typically do not proliferate, (2) those that may proliferate and remain with the host (e.g., on the skin or in the GI tract) for relatively short periods of time, and (3) those that may permanently colonize the host.

It has been recognized that the health of a host depends at least in part on the health of the microbiome of the host. For example, the health benefits provided by certain microorganisms typically found in a human GI tract have been well studied. Similarly, the undesirable effects of an unhealthy or unbalanced GI microbiome are also well known. The knowledge of the relationship between the health of a host and the health of the GI microbiome of the host has led to a variety of commercially available products marketed to improve or maintain the health of one or more members of the human GI microbiome. These commercially available products are generally classified as probiotics, prebiotics or synbiotics. Probiotics are so-called “good” microorganisms (typically bacteria) that are ingested alive by a person so that the introduced microorganisms can colonize the GI tract of the person. Conventional prebiotics are ingestible ingredients that selectively support the growth or survival of the “good” microorganisms which are desirably present in the GI tract. Conventional prebiotics are typically a nutrient source (e.g., fructooligosaccharide or galactooligosaccharide) that can be assimilated by one or more members of the GI microbiome, but which are not digestible by the human host. Synbiotics are a mixture of prebiotic and probiotic. The prebiotic portion of the synbiotic provides a suitable nutrient source to the probiotic portion of the symbiotic, which is believed to increase the likelihood of probiotic survival and colonization.

More recently, attention has turned to the microflora found on human skin to better understand the relationship between the health of the resident microflora and the health of the host. Not surprisingly, it has been found that a healthy balanced skin microbiome can provide health and/or cosmetic benefits to the human host, for example, by stimulating the human immune system and/or producing anti-microbial substances targeted at reducing colonization of unwanted microorganisms. On the other hand, perturbations that disrupt the delicate balance of the skin microflora may result in undesirable consequences to the host and/or microflora. For example, increased production of free fatty acid byproducts associated with the proliferation of Propionibacterium acnes may promote the development of acne. The makeup of the human skin microbiome differs significantly from the makeup of the GI microbiome in terms of both the type and variety of microorganisms present. Thus, it may come as no surprise that the members of the GI and skin microbiomes may utilize different nutrient sources due to, at least in part, the starkly contrasting environments in which the two microbiomes are found and the substrates available for use as food.

It is well known that the dietary requirements of microorganisms can vary significantly from one species to the next, and it is not uncommon for an agent that exhibits prebiotic activity on a particular microorganism to exhibit no prebiotic activity on a different microorganism. For example, prebiotics designed for the GI microbiota have historically been carbohydrate-based materials that serve as food for the resident glycolytic driven microorganisms. But the microflora present on the skin of a person can include lipophilic organisms, which would not necessarily be expected to assimilate carbohydrates. Even the glycolytic microorganisms which may be present on the skin may not utilize the same kinds of carbohydrates as the GI microbes, since the microorganisms present on the skin are generally not exposed to the same kinds of carbohydrates as the microorganisms in the GI tract.

While it may come as no surprise that the make up of the GI and skin microbiomes of a human may vary significantly, perhaps more surprising is the finding that there can also be significant variability in the make up of the same microbiome between individuals. The health and cosmetic benefits of providing a healthy, balanced skin microbiome are only recently becoming better understood. As a result, only a limited number of suitable prebiotic agents have been identified for use on skin. In addition, conventional prebiotic agents are typically administered orally, for example, as part of a nutritional supplement regimen. While oral ingestion may be suitable for delivering prebiotic agents to the GI tract, it may not be the best way to deliver a prebiotic to the microbiota found on the skin.

Accordingly, there is a need to improve the health and/or appearance of human skin by providing an agent that exhibits prebiotic activity on one or more skin commensal microorganisms. There is also a need for an improved mechanism of delivering a prebiotic agent to skin commensal microorganisms.

### SUMMARY OF THE INVENTION

In order to provide a solution to one or more of the problems above, disclosed herein is a method for improving the condition and/or appearance of skin. The method comprises topically applying a cosmetic composition to the skin. The cosmetic composition comprises a dermatologically acceptable carrier and a galactooligosaccharide.

### BRIEF DESCRIPTION OF THE DRAWINGS

FIG. 1 illustrates an exemplary microbiome population distribution.

FIG. 2 illustrates microbial ATP response to a test agent over the course of time based on in vitro testing.

FIG. 3 illustrates bacterial count response to a test agent over the course of time based on in vitro testing.

FIG. 4 illustrates microbial ATP response to various levels of a test agent based on in vitro testing.

FIG. 5 illustrates bacterial count response to various levels of a test agent based on in vitro testing.

FIG. 6 illustrates bacterial count response of aerobic microbes to a test agent based on in vivo testing.

FIG. 7 illustrates bacterial count response of anaerobic microbes to a test agent based on in vivo testing.

FIG. 8 illustrates in vitro bacterial ATP response to a variety of compositions.

FIG. 9 shows portions of the test schedule for an in vivo study.

FIG. 10 illustrates exemplary positions of test areas on the forearm of a person.

### DETAILED DESCRIPTION OF THE INVENTION

Definitions

“Cosmetic composition” means a composition suitable for topical application on mammalian skin and/or other keratinous tissue such as hair and nails, which is intended to improve the condition and/or appearance of the skin or keratinous tissue or otherwise provide a skin care benefit. Topical means the surface of the skin or other keratinous tissue. Cosmetic composition includes any color cosmetic, nail, or skin care product. “Skin care” means regulating and/or improving skin condition. Some nonlimiting examples of skin care benefits include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin texture or smoothness, improving skin exfoliation or desquamation, plumping the skin, improving skin barrier properties, improve skin tone, reducing the appearance of redness or skin blotches, and/or improving the brightness, radiancy, or translucency of skin. Some non-limiting examples of cosmetic compositions include products that leave color on the face, such as foundation, mascara, concealers, eye liners, brow colors, eye shadows, blushers, lip sticks, lip balms, face powders, solid emulsion compact, and the like. “Skin care products” include, but are not limited to, skin creams, moisturizers, lotions, and body washes.

“Dermatologically acceptable carrier” means a carrier that may be applied topically to skin or keratinous tissue. The dermatologically acceptable carrier may be in a wide variety of forms such as, for example, simple solutions (water-based or oil-based), solid forms (gels or sticks) and emulsions (water-in-oil or oil-in-water).

“Effective amount” means a sufficient amount of the specified component to have the specified properties under the specified conditions. For example, an effective amount of a prebiotic means an amount sufficient to cause a desired increase in the metabolite level and/or bacterial counts of one or more selected microorganisms in vitro and/or in vivo.

“Gastrointestinal microorganisms” or “GI microorganisms” are prokaryotes and/or eukaryotes that colonize (i.e., live and multiply) in the human digestive tract.

“Increase” means increases above basal levels, or as compared to a control. For example, basal levels may be determined for in vivo studies while a control is used for in vitro tests.

“Metabolism” means any chemical reaction occurring inside a microorganism. Metabolism includes anabolism, the synthesis of the biological molecules (e.g. protein synthesis and DNA replication) and catabolism, the breakdown of biological molecules.

“Microbial lysate” means the mixture of cellular components and reagents that result from the lysis of a microorganism. “Lysis” involves the action of rupturing the cell wall and/or the cell membrane of a cell by a treatment (e.g. chemical, biological, mechanical, or thermal treatment), resulting in the release of some or all of the cell's biological constituents.

“Microorganism” and “microbe” are synonymous and mean bacteria, fungi, and algae.

“Minimal carbon medium” (“MCM”) means a mixture of substances used to support the limited growth (i.e., less than a 0.2 log increase in colony forming units (“CFU”) in a 24 hour period) and/or survival of microorganisms in which carbon is a limiting resource. In certain embodiments, the MCM may be in the form of a liquid or a gel. Because the minimum carbon requirements may vary between different microorganisms, the amount of carbon present in the MCM may also vary. In certain embodiments, for example, the MCM may be completely free of carbon. In certain embodiments, the MCM may be substantially free of carbon (i.e., less than 0.001% by weight based on the weight of the medium). In certain embodiments, the MCM may contain from 0.001% to 0.1% of carbon. The amount of carbon is determined as the mole fraction or molecular weight % of carbon present. For example, glucose is 40% carbon by weight.

“Oligosaccharide” means a saccharide polymer containing a small number (e.g., two to ten) of monosaccharides.

“Orally ingestible” refers to compositions that are intended to be placed in the mouth and swallowed.

“PCR” means polymerase chain reaction and includes real-time PCR, quantitative PCR (“QPCR”), semi-quantitative PCR, and combinations thereof.

“Prebiotic” means any substance or combination of substances that can be utilized as a nutrient by a selected microorganism (e.g., a skin commensal microorganism or a GI microorganism), can induce the growth and/or activity of a selected microorganism, can induce the replication of a selected microorganism, can be utilized as an energy source by a selected microorganism, and/or can be utilized by a selected microorganism for the production of biomolecules (i.e. RNA, DNA, and proteins). Non-limiting examples of prebiotics include mucopolysaccharides, oligosaccharides such as galactooligosaccharides (“GOS”), polysaccharides, amino acids, vitamins, nutrient precursors, harvested metabolic products of biological organisms, lipids, and proteins. In order to determine whether a test agent exhibits prebiotic activity on a microorganism, it may be desirable to combine the test agent with an inert buffer (e.g., saline) or a solvent. Non-limiting examples of suitable solvents include dimethylsulfoxide (DMSO), alcohols such as methanol and ethanol, and aqueous solutions such as water and culture medium.

“Replication” means the division of a microorganism into daughter cells (e.g. by mitosis or binary fission).

“Skin” means one or more of the epidermis, dermis, and hypodermis (i.e., subcutis), hair follicles, hair roots, hair bulbs, the ventral epithelial layer of the nail bed (lectulus), sebaceous glands and perspiratory glands (eccrine and apocrine).

“Skin commensal microorganisms” means prokaryotes and eukaryotes that may colonize (i.e., live and multiply on human skin) or temporarily inhabit human skin in vitro and/or in vivo. Exemplary skin commensal microorganisms include, but are not limited to, Alphaproteobacteria, Betaproteobacteria, Gammaproteobacteria, Propionibacteria, Corynebacteria, Actinobacteria, Clostridiales, Lactobacillales, Staphylococcus, Bacillus, Micrococcus, Streptococcus, Bacteroidales, Flavobacteriales, Enterococcus, Pseudomonas, Malassezia, Maydida, Debaroyomyces, and Cryptococcus.

“Topical” and variations thereof refer to compositions that are intended to be applied directly to the outer surface of the skin or other keratinous tissue.

The articles “a” and “an” are understood to mean one or more of what is being claimed and/or described.

Selection of Target Microorganism(s)

The surface of mammalian skin typically includes a wide variety of microorganisms, which may vary from species to species, individual to individual, and even from location to location on an individual. Collectively, these microorganisms form a microbiome. A healthy skin microbiome will generally consist of a balanced collection of skin commensal microorganisms. The skin microbiome of a human host may include a variety of resident microorganisms that help promote the health and/or appearance of the host's skin. But in some instances, certain undesirable microorganisms such as pathogenic bacteria, yeasts and molds may attempt to colonize the skin. Colonization by such microorganisms can upset the balance of a healthy microbiome. Fortunately, the resident microorganisms typically (and desirably) present in the human skin microbiome have evolved a variety of active and passive mechanisms to inhibit and/or prevent colonization of the skin by undesirable microorganisms. Examples of the passive mechanisms include competing for niches that can be occupied by undesirable microorganisms and consuming nutrients essential for the growth and proliferation of undesirable microorganisms. In terms of active mechanisms, desirable microorganisms may produce metabolites that inhibit the proliferation of undesirable microorganisms, or even kill them outright. In addition to inhibition of undesirable microorganisms, there is a growing body of evidence that certain resident microflora impact innate immunity. For example, it has been demonstrated that certain members of the skin microbiome via their metabolism of lipids, proteins and carbohydrates, produce acid that aids in maintaining the “acid mantel” of the skin.

One approach to maintaining a microbiome in a healthy, balanced state and/or returning a microbiome to a healthy, balanced state may be to provide certain desirable microorganisms with sufficient nutrients to thrive, and thereby outcompete and/or kill the undesirable bacteria. For example, it may be desirable to include one or more prebiotic agents in the compositions used by a person in their daily skin care regimen. However, this is not an easy task because the variability in the makeup of the microorganisms from person to person may render a particular agent suitable as an effective prebiotic for the skin commensal microorganism of one person but not another. Notwithstanding the wide variability that may be observed in the skin commensal microorganisms of different individuals, it has been found that some commonalities do exist. For example, it has been found that Corynebacterium jeikeium (“C. jeikeium”), Staphylococcus epidermidis (“S. epidermidis”), and Propionibacterium acnes (“P. acnes”) to varying extents are present in measurable quantities on both the face and forearms of humans.

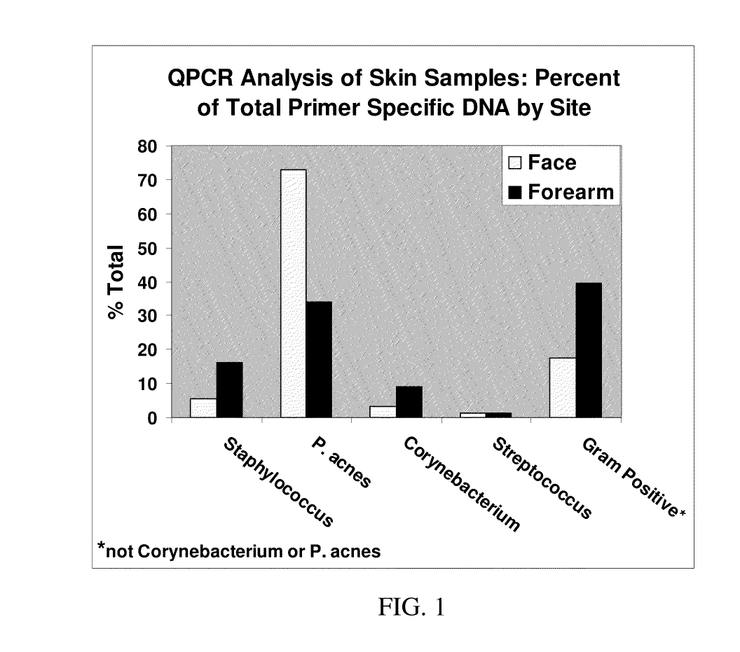

FIG. 1 illustrates the similar yet diverse microbial populations that may be present on the face and forearm of a person. The microorganisms illustrated in FIG. 1 were isolated by sampling the skin with a sterile swab wetted with phosphate buffered saline (“PBS”). The QPCR analysis illustrated in FIG. 1 utilized DNA isolated from the swab samples. As shown in FIG. 1, Staphylococcus, Corynebacterium and Propionibacterium are all present on the face and forearm of the individuals sampled. Thus, the inclusion of P. acnes, Staphylococcus and Corynebacterium in a prebiotic screening method may be particularly useful for predicting the in-vivo effect of a potential prebiotic agent. FIG. 1 also illustrates that Propionibacterium may be more commonly found on the face than the forearm, while the opposite appears to be true for Corynebacterium and Staphylococcus. Thus, an agent that exhibits prebiotic activity for P. acnes may potentially have a robust impact on skin health and/or the skin microbiome due to their proportionate contribution to the makeup of the forearm and face microbiomes. And an agent that exhibits prebiotic activity for Corynebacterium and Staphylococcus may be used to provide a targeted skin health benefit specific to the forearms and/or other bodily regions that have a similar microbiome make up.

With regard to skin commensal microorganisms which may desirably affect the skin microbiome and/or skin health, it is believed that C. jeikeium, S. epidermidis, and P. acnes provide a skin health and/or desirable microbiome benefit, which may be increased by providing these microbes with a compound having prebiotic potential. In particular, it has been demonstrated that C. jeikeium produces siderophores that sequester iron. C. jeikeium also employs specialized mechanisms for acquiring manganese, both of which are essential for the growth of certain undesirable microorganisms.

S. epidermidis is believed to play an active role in stimulating the immune system of the skin, for example, by influencing the innate immune response of keratinocytes through Toll-like receptor (“TLR”) signaling. Additionally, S. epidermidis is believed to occupy receptors on a host cell that are also recognized by more virulent microorganisms such as Staphylococcus aureus. Further, S. epidermidis produces lanthionine-containing antibacterial peptides, sometimes referred to as bacteriocins, which are known to exhibit antibacterial properties toward certain species of harmful bacteria. Examples of such peptides include: epidermin, epilancin K7, epilancin 15X Pep5, and staphylococcin 1580. Other peptides produced by S. epidermidis counteract intra- and interspecies competitors. The peptides are effective against Streptococcus aureus, group A streptococcus, and Streptococcus pyogenes.

P. acnes is a commensal, non-sporulating bacilliform (rod-shaped), gram-positive bacterium found in a variety of locations on the human body including the skin, mouth, urinary tract and areas of the large intestine. P. acnes can consume skin oil and produce by-products such as short-chain fatty acids and propionic acid, which are known to help maintain a healthy skin pH and barrier properties. Propionibacteria such as P. acnes also produce bacteriocins and bacteriocin-like compounds (e.g., propionicin P1G-1, jenseniin G, propionicins SM1, SM2 T1, and acnecin), which are inhibitory toward undesirable lactic acid-producing bacteria, gram-negative bacteria, yeasts, and molds.

Considering the beneficial functions believed to be provided by C. jeikeium, S. epidermidis and P. acnes and the presence they appear to have on both the forearms and face of a person, it would be desirable to provide agents that exhibit suitable in vivo prebiotic activity for one, two, or even all of these skin commensal microorganisms. And since at least some cosmetic compositions are commonly applied to the face, hands and/or forearms of a person, it may be desirable to incorporate ingredients into these cosmetic compositions that promote the health and/or survival of C. jeikeium, S. epidermidis, and/or P. acnes. Of course, it is to be understood that the prebiotic activity described herein is not limited to the foregoing microorganisms, but may exhibit suitable prebiotic activity on other skin commensal microorganisms as well.

Prebiotic Agent

Microorganisms, and indeed all life forms, have evolved to be successful in their environment. One aspect of the evolution of an organism is adapting to utilize available food sources commonly found in the organism's habitat. Thus, skin commensal microorganisms tend to utilize nutrient sources commonly found on and/or in the skin, while microorganisms that populate the GI tract tend to utilize food sources commonly found in the GI tract. For example, P. acnes, which is present on the skin of most humans, is known to consume fatty acids in the sebaceous glands or sebum secreted by hair follicles. On the other hand, Bifidobacterium bifidum, which is commonly found in the GI tract of humans, can utilize galactooligosaccharides (“GOS”) as a food source. Because of the substantial differences between the environments in the GI tract and on the skin and the available nutrients commonly found in each environment, skin commensal microbes and GI microbes are not expected to utilize the same food sources.

Ingestible forms of prebiotic agents such as GOS are well known for improving the health of the GI microbiome. As indicated previously, Bifidobacterium bifidum, which is generally considered to be a beneficial species of bacteria found in the human GI tract, is known to use GOS as a food source. GOS are galactose-containing oligosaccharides commonly produced from lactose using the transgalactosylase activity of the enzyme β-galactosidase. Depending on the method used to make it, GOS may include di-, tri-, tetra-, penta-, or hexa-saccharides or a mixture of two or more of these according to the following formula:

Glc \acute{α}1-1[β-Gal 1-6]ₙ
where n=2-5,
Gal represents a galactose residue and
Glc represents a glucose residue.
   In a particularly suitable embodiment, the GOS may be in form of a mixture that includes from 20 to 35% w/v of a disaccharide, from 20 to 35% w/v of a trisaccharide, from about 15 to about 25% w/v of a tetrasaccharide, and from the 10 to 20% w/v of a pentasaccharide. U.S. Pat. No. 7,883,874 to Gibson, et al. and U.S. Pat. Nos. 8,030,049 and 8,058,047 to Tzortzis, et al., each disclose examples of GOS and methods of making GOS.

GOS are commercially available in a variety of forms such as powders and syrups. GOS may also be found as ingredients in food products sold for human and/or animal consumption. A particularly suitable example of a commercially available source of GOS is BIMUNO, available from Clasado, Inc., Panama. It is believed that BIMUNO is a mixture of GOS, dietary fiber and other filler ingredients. U.S. Pat. No. 7,883,874 to Gibson, et al., discloses a suitable example of GOS produced by a strain of B. bifidum that converts lactose to the aforementioned mixture of GOS by way of galactosidase enzyme activity. The GOS produced in this way are described as including at least one disaccharide, at least one trisaccharide, at least one tetrasaccharide and at least one pentasaccharide. While GOS are known prebiotic agents for GI microorganisms, GOS are typically not found on human skin in significant amounts. As a result, GOS have not been previously considered for use as a prebiotic for skin commensal microorganisms. However, it has surprisingly been found that GOS exhibit a desirable level of prebiotic activity on at least some skin commensal microorganisms. In particular, GOS exhibit prebiotic activity for C. jeikeium, S. epidermidis and P. acnes.

While the foregoing example describes GOS as suitable skin commensal prebiotic agents, it is to be appreciated that other GI prebiotics, but not all GI prebiotics, as discussed in more detail below, may be suitable for use as skin commensal prebiotic agents. Some non-limiting examples of GI prebiotics that may be suitable for use as skin commensal prebiotics include hydroxyisoleucine; wheat dextrin; arabinogalactan (e.g., larch arabinogalactin); citrus fiber; pea fiber; maltodextrin; oligofructose (i.e., fructooligosaccharides or “FOS”); inulin; inulin oligofiber; mannan hydrolysates; glucomannan hydrolysates; galactomannan; gentiooligosaccharides; isomaltooligosaccharide; kimi and kiwi derived compounds (e.g., ZYACTINASE 45 brand enzyme complex derived from kiwi and available from Vital Foods); beet pulp; and rice bran.

To be suitable for use as a prebiotic for a skin commensal microorganism, the composition or agent should promote the survival and/or growth of the microorganism. In order to determine the prebiotic potential of a test agent, it may be desirable to measure a metabolite formed as a result of exposing a skin commensal microorganism to the test agent. Suitable microbial outputs include, without limitation, levels of metabolites such as ATP, NAD, NADP, NADH, NADPH, cAMP, cGMP, and/or ADP), which are released upon cell lysis. In some instances, the metabolic indicators may be measured with a commercially available enzyme-based assay. Additionally or alternatively, it may be desirable to measure the change in number and/or concentration of the microorganism(s) (i.e., proliferation) to determine if prebiotic activity is exhibited. For example, an increase in bacterial counts (e.g., when measured by a suitable plate count test) may be sufficient to demonstrate prebiotic activity.

In vivo testing is generally preferred for determining prebiotic activity. But such testing can be time consuming and expensive. Conventional in vitro testing (e.g., ATP assay or plate count), while typically faster and less expensive than in vivo testing, may not provide a suitable prediction of in vivo activity. Thus, it may be desirable to use a tiered approach in which one or more types of in vitro testing are used to predict whether the GOS will exhibit prebiotic activity in vivo, optionally followed by in vivo testing to confirm such activity. Particularly suitable examples of tiered screening assays and methods for determining prebiotic activity are disclosed in co-pending U.S. Ser. Nos. 13/672,163; 13/672,192; and 13/672,211 all filed by Lanzalaco, et al.

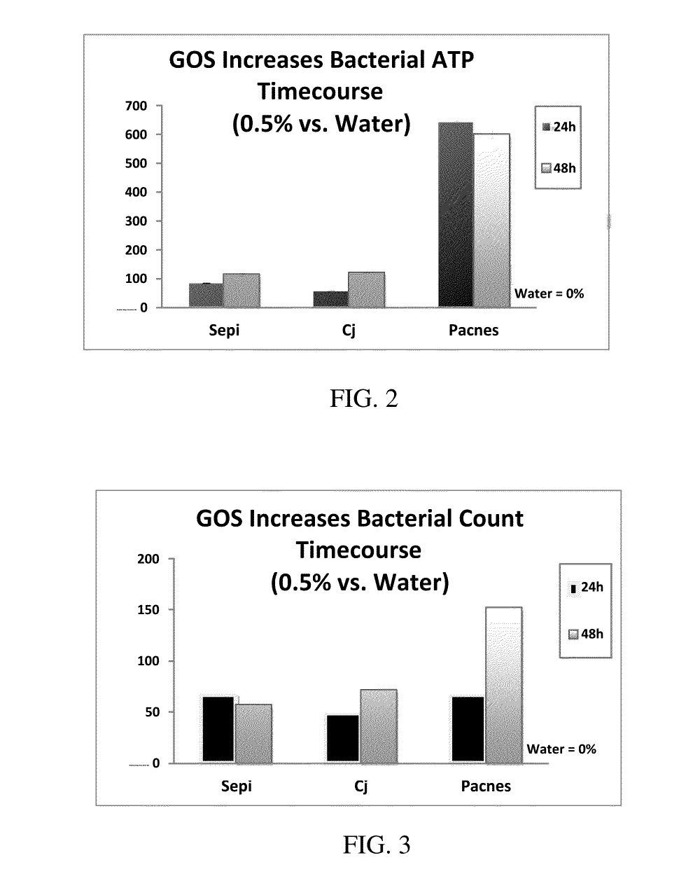

FIGS. 2 and 3 illustrate the in vitro prebiotic effect of GOS versus time when present at an amount of 0.5% by weight based on the volume of the test sample. FIG. 2 illustrates the percent change in ATP production of three skin commensal microorganisms relative to a water control at 24 hours and 48 hours. The three skin commensal microorganisms illustrated in FIGS. 2 and 3 are S. epidermidis (shown as “Sepi”), C. jeikeium (shown as “Cj”) and P. acnes (shown as “Pacnes”). As illustrated in FIG. 2, the ATP production of all three skin commensal microorganisms increases relative to the water control at 24 and 48 hours. The ATP level is determined according to the ATP Test described in more detail below. FIG. 3 illustrates the percent change in bacterial count of the three skin commensal microorganisms relative to a water control when measured at 24 hours and 48 hours. The bacterial count is measured by the Plate Count Test described in more detail below. As illustrated in FIG. 3, the bacterial counts increase at 24 and 48 hours relative to the water control. In other words, the GOS exhibited prebiotic activity in vitro at 24 and 48 hours for the microbes tested. The test samples used to generate the data illustrated in FIGS. 2 and 3 are prepared according to the method described below for creating starter cultures, work cultures and test samples. The test samples are a mixture of BIMUNO brand GOS, minimal carbon medium, and the selected microorganism.

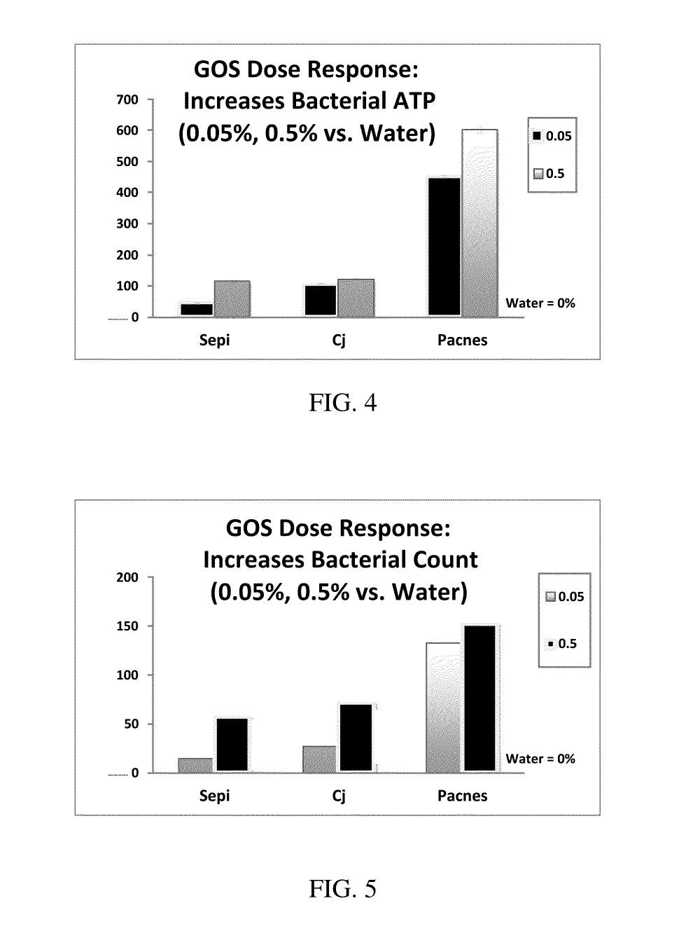

FIGS. 4 and 5 illustrate the comparative in vitro prebiotic effect of GOS at 0.05% and 0.5% by weight based on the volume of the test sample. As illustrated FIG. 4, the ATP production of all three skin commensal microorganisms increases relative to the water control at both the 0.05% and 0.5% levels. FIG. 5 illustrates an increase in bacterial counts at the 0.05% and 0.5% levels. Thus, the GOS exhibited prebiotic activity in vitro when present at 0.05% and 0.5%. The test samples used to generate the data illustrated in FIGS. 4 and 5 are prepared according to the method described below for creating starter cultures, work cultures and test samples. The test samples are a mixture of BIMUNO brand GOS, minimal carbon medium, and the selected microorganism.

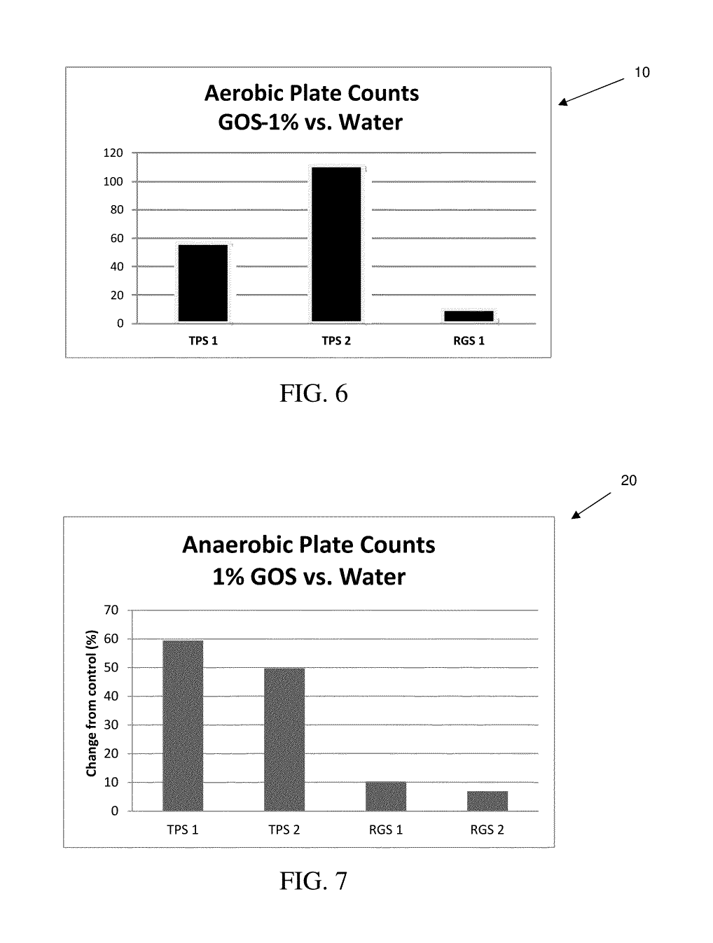

FIG. 6 illustrates the in vivo prebiotic effect of GOS on at least some of the aerobic microorganisms in the skin microbiome, when the microorganisms are exposed to a 1% GOS test sample by weight based on the volume of the test sample. The chart 10 illustrates aerobic bacterial counts that correspond to samples taken from human test subjects during an in vivo clinical study, which is described in more detail below. The samples shown as TPS 1 and TPS 2 in the chart 10 correspond to microbial samples taken during the Treatment Phase of the study, in which the 1% GOS test sample is present on the forearm of the test subjects. The sample shown as RGS 1 in the chart 10 corresponds to the first microbial sample taken during the Regression Phase of the study, in which GOS are not present on the forearm. As illustrated in FIG. 6, the aerobic bacterial counts increased during the Treatment Phase relative to the baseline level measured during an initial Conditioning Phase, which is described in more detail below, and decreased during the Regression Phase relative to the Treatment Phase. Based on the data shown in FIG. 6, it is believed that the GOS present during the Treatment Phase resulted in the increase in aerobic bacterial counts, and that the subsequent lack of GOS during the Regression Phase resulted in the decrease in aerobic bacterial counts. In other words, the GOS exhibited prebiotic activity in vivo on at least some aerobic skin commensal microorganisms when present at 1%. The test samples used to generate the data illustrated in FIG. 6 are aqueous solutions of 1% BIMUNO brand GOS.

FIG. 7 illustrates the in vivo prebiotic effect of GOS on at least some of the anaerobic microorganisms in the skin microbiome, when the microorganisms are exposed to a 1% GOS test sample. TPS 1, TPS 2, and RGS 1 correspond to the same sample times as described with regard to FIG. 6. RGS2 corresponds to the second microbial sample taken during the Regression Phase. As can be seen in FIG. 7, the anaerobic bacterial counts increased during the Treatment Phase relative to the baseline level measured during the Conditioning Phase and decreased during the Regression Phase relative to the Treatment Phase. Additionally, FIG. 7 illustrates the continued decrease in anaerobic bacterial counts in RGS2 relative to RGS1. Based on the data illustrated in the chart 20 of FIG. 7, it is believed that the GOS present during the Treatment Phase resulted in the increase in anaerobic bacterial counts, and that the subsequent lack of GOS during the Regression Phase resulted in the decrease in anaerobic bacterial counts. In other words, the GOS exhibited prebiotic activity in vivo on at least some anaerobic skin commensal microorganisms when present at 1%. The test samples used to generate the data illustrated in FIG. 7 are aqueous solutions of 1% BIMUNO brand GOS.

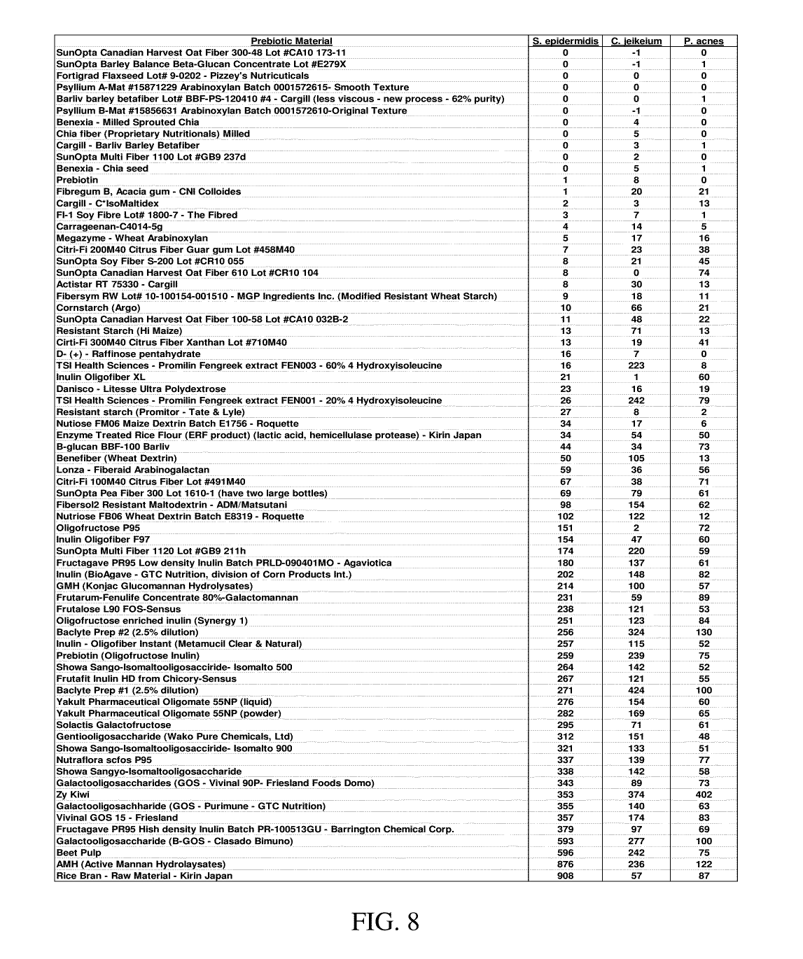

While it has been surprisingly found that certain GI prebiotics exhibit suitable prebiotic potential for skin commensal microorganisms, the same is not true for all commonly known GI prebiotics, even those that are similar in composition to GOS (i.e., carbohydrate-based). FIG. 8 illustrates the prebiotic potential of a variety of carbohydrate-based, GI prebiotics for S. epidermis, C. jeikeium and P. acnes by measuring the change in bacterial ATP levels relative to a water control. As can be seen in FIG. 8, not all the GI prebiotics exhibit desirable prebiotic potential for the three skin commensal microorganisms. The test samples used to generate the data illustrated in FIG. 8 were prepared according to the method described below for creating starter cultures, work cultures and test samples. The test samples include one of the test agents shown in FIG. 8 present at 1% by weight based on the volume of the test sample. The test samples are a mixture of test agent, minimal carbon medium, and the selected microorganism.

### Cosmetic Compositions.

It is believed, without being limited by theory, that the health of the skin microbiome may be linked to desirable skin function or appearance and/or may otherwise provide one or more skin care benefits. For example, it may be possible to maintain or improve the appearance, barrier function, moisture retention and/or other properties of skin by maintaining or improving the health of one or more members of the skin microbiome. In some instances, if a particular area or areas of the skin exhibit undesirable function and/or appearance it may be desirable to target that particular area or areas of the skin for maintenance or improvement. For example, it may be desirable to target particular areas of the skin such as on the face (e.g., forehead, cheeks, and peri-orbital portions of the face), hands and/or forearms, which tend to be more damaged by exposure to the environment (e.g., UV radiation, wind, pollution, oxidation, irritants) than some other areas of the skin and/or which may be subject to visible signs of intrinsic aging. Topically applied cosmetic compositions for improving the health and/or appearance of skin are well known (e.g., lotions, moisturizing creams, oils, foundations (liquid and powder), lipsticks, concealers, shave prep compositions, liquid or solid cleansing soaps). Thus, it may be desirable to incorporate prebiotic agents such as GOS into topical cosmetic compositions to exploit the health and/or appearance benefit(s) that may be provided by a healthy, balanced skin microbiome.

The cosmetic compositions herein may include an effective amount of a skin commensal prebiotic agent. The prebiotic agent may be present at an amount of greater than 0.001%, 0.01%, 0.05%, 0.1%, 0.5%, 1%, 2%, 3%, 4% or even greater than 5% by weight of the composition. It may be desirable to limit the amount of the prebiotic agent in the present cosmetic compositions to an amount of less than 25%, 20%, 15%, or even 10% by weight of the composition to avoid cosmetically undesirable characteristics (e.g., stickiness or poor spreadability). In certain embodiments, the prebiotic agent may be present at an amount sufficient to increase the ATP level of at least one skin commensal microorganism by at least 80% (e.g., from 80-1000% or more or any value in this range) in vitro. Additionally or alternatively, the prebiotic agent may be present at an amount sufficient to increase the ATP level of at least two skin commensal microorganisms by at least 50% (e.g., from 50-1000% or more or any value in this range) in vitro. Further, the prebiotic agent may be present at an amount sufficient to increase the ATP level of at least three skin commensal microorganisms by at least 25% (e.g., from 25-1000% or more or any value in this range) in vitro. It is to be appreciated that the prebiotic may be present in the composition at an amount that provides one or more of the above increases in ATP level in vitro. For example, the prebiotic agent may be present at an amount sufficient to increase the ATP level counts of a first skin commensal microorganism by at least 80% in vitro and the ATP level of a second skin commensal microorganism by at least 50% in vitro. Continuing with this example, the prebiotic agent may also be present at an amount sufficient to increase the ATP level of a third skin commensal microorganism by at least 25% in vitro. The ATP level may be determined in vitro according to the ATP Test described in more detail below.

In certain embodiments, the prebiotic agent may be present at an amount sufficient to increase the bacterial counts of at least one skin commensal microorganism by at least 10% in vitro (e.g., from 10-200% or more, 50-175%, 100-150%, or any value in these ranges). Additionally or alternatively, the prebiotic agent may be present at an amount sufficient to increase the bacterial counts of at least two skin commensal microorganisms by at least 10% in vitro (e.g., from 10-200%, 20-180%, 30-160%, 40-150%, 50-120%, or any value in these ranges). Further, the prebiotic agent may be present at an amount sufficient to increase the bacterial counts of at least three skin commensal microorganisms by at least 10% in vitro (e.g., from 10-200% or more or any value in this range). It is to be appreciated that the prebiotic may be included in the present compositions at an amount that provides one or more of the above increases in bacterial counts in vitro. For example, the prebiotic agent may be present at an amount sufficient to increase the bacterial counts of a first skin commensal microorganism by at least 50% in vitro and the bacterial counts of a second skin commensal microorganism by at least 20% in vitro. Continuing with this example, the prebiotic agent may also be present at an amount sufficient to increase the bacterial counts of a third skin commensal microorganism by at least 10% in vitro. The in vitro bacterial counts may be determined according the Plate Count Test described in more detail below.

The present cosmetic compositions desirably include a prebiotic agent at an amount sufficient to increase the in vivo bacterial counts of at least one aerobic and/or anaerobic skin commensal microorganism (e.g., one or more of the skin commensal microorganisms described hereinabove). In certain embodiments, the prebiotic agent may be present at an amount to increase the aerobic and/or anaerobic in vivo bacterial counts by at least 10% (e.g., at least 20%, 30%, 40%, 50%, 60%, 70%, 80%, 90%, 100%, 110%, 120%, 130% or more or any value within these ranges), but less than a 100× increase (e.g., less than 90×, 80×, 70×, 60×, 50, 40×, 30, 20×, 10×, or 5×). The present cosmetic compositions desirably include the prebiotic agent in an amount sufficient to provide a skin care benefit.

In certain embodiments, the cosmetic composition may include a dermatologically acceptable carrier, an effective amount of a skin commensal prebiotic, and one or more optional ingredients of the kind commonly included in the particular cosmetic compositing being provided.

Dermatologically Acceptable Carriers

In certain embodiments, the cosmetic compositions herein may include one or more suitable carriers in the form of water and/or water miscible solvents. The carrier may be present at an amount of from 1% to 99% by weight, based on the weight of the composition (e.g., from 1%, 3%, 5%, 10%, 15%, 20%, 25%, 30%, 35%, 40%, 45%, 50%, 55%, 60%, 65%, 70%, 75%, 80%, or 85% to 90%, 85%, 80%, 75%, 70%, 65%, 60%, 55%, 50%, 45%, 40%, 35%, 30%, 25%, 20%, 15%, 10%, or 5%). Suitable water miscible solvents include monohydric alcohols, dihydric alcohols, polyhydric alcohols, glycerol, glycols, polyalkylene glycols such as polyethylene glycol, and mixtures thereof. When the cosmetic composition is in the form of an emulsion, the water and/or water miscible solvents are typically associated with the aqueous phase of the emulsion.

The cosmetic compositions herein may include one or more suitable oils. The oils may be volatile or nonvolatile oils. Volatile oils suitable for use herein may have a viscosity ranging from 0.5 to 5 centistokes (cSt) at 25° C. Volatile oils may be used to promote more rapid drying of the skin care composition after it is applied to skin. Nonvolatile oils may be included to provide emolliency and protective benefits to the skin.

In certain embodiments, the cosmetic compositions may include one or more suitable silicone oils such as, for example, one or more polysiloxanes. Polylsiloxanes suitable for use herein may have a viscosity of from 0.5 to 1,000,000 centistokes at 25° C. and can be represented by the general chemical formula:

R₃SiO[R₂SiO]ₓSiR₃

wherein R is independently selected from hydrogen or C₁₋₃₀ straight or branched chain, saturated or unsaturated alkyl, phenyl or aryl, trialkylsiloxy; and x is an integer of from 0 to 10,000, chosen to achieve the desired molecular weight. In certain embodiments, R is hydrogen, methyl, or ethyl. Commercially available polysiloxanes include the polydimethylsiloxanes, which are also known as dimethicones, examples of which include the DM-Fluid series from Shin-Etsu, the Vicasil® series sold by Momentive Performance Materials Inc., and the Dow Corning® 200 series sold by Dow Corning Corporation. Specific examples of suitable polydimethylsiloxanes include Dow Corning® 200 fluids (also sold as Xiameter® PMX-200 Silicone Fluids) having viscosities of 0.65, 1.5, 50, 100, 350, 10,000, 12,500 100,000, and 300,000 cSt.

Suitable dimethicones include those represented by the general chemical formula:

R₃SiO[R₂SiO]ₓ[RR′SiO]_{y}SiR₃

wherein R and R′ are each independently hydrogen or C₁₋₃₀ straight or branched chain, saturated or unsaturated alkyl, aryl, or trialkylsiloxy; and x and y are each integers of 1 to 1,000,000 selected to achieve the desired molecular weight. Suitable dimethicones include phenyl dimethicone (Botansil™ PD-151 from Botanigenics, Inc.), diphenyl dimethicone (KF-53 and KF-54 from Shin-Etsu), phenyl trimethicone (556 Cosmetic Grade Fluid from Dow Corning), or trimethylsiloxyphenyl dimethicone (PDM-20, PDM-200, or PDM-1000 from Wacker-Belsil). Other examples include alkyl dimethicones wherein at least R′ is a fatty alkyl (e.g., C₁₂₋₂₂). A suitable alkyl dimethicone is cetyl dimethicone, wherein R′ is a straight C16 chain and R is methyl. Cetyl dimethicone, is available as s 2502 Cosmetic Fluid from Dow Corning or as Abil Wax 9801 or 9814 from Evonik Goldschmidt GmbH.

Other silicone oils that may be suitable for use in the cosmetic compositions herein include cyclic silicones having the general formula:

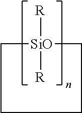

wherein R is independently selected from hydrogen or C₁₋₃₀ straight or branched chain, saturated or unsaturated alkyl, phenyl or aryl, trialkylsiloxy; and where n=3-8 and mixtures thereof. Commonly, a mixture of cyclomethicones is used where n is 4, 5, and/or 6. Commercially available cyclomethicones include Dow Corning UP-1001 Ultra Pure Fluid (i.e. n=4), Dow Corning XIAMETER® PMX-0245 (i.e. n=5), Dow Corning XIAMETER® PMX-0245 (i.e. n=6), Dow Corning 245 fluid (i.e. n=4 and 5), and Dow Corning 345 fluid (i.e. n=4, 5, and 6).

In certain embodiments, hydrocarbon oils (e.g., straight, branched, or cyclic alkanes and alkenes) may be included in the present cosmetic compositions. The chain length of the hydrocarbon oil may be selected based on the desired functional characteristics such as volatility. Suitable volatile hydrocarbons may have between 5-20 carbon atoms or, alternately, between 8-16 carbon atoms.

Other oils that may be suitable for use in the present cosmetic compositions include esters of at least 10 carbon atoms. These esters include esters with hydrocarbyl chains derived from fatty acids or alcohols (e.g., mono-esters, polyhydric alcohol esters, and di- and tri-carboxylic acid esters). The hydrocarbyl radicals of the esters hereof may include or have covalently bonded thereto other compatible functionalities, such as amides and alkoxy moieties (e.g., ethoxy or ether linkages, etc.). Exemplary esters include, but are not limited to, isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, C₁₂₋₁₅ alkyl benzoate, diisopropyl adipate, dibutyl adipate, and oleyl adipate. Other suitable esters are further described in the Personal Care Product Council's International Cosmetic Ingredient Dictionary and Handbook, Thirteenth Edition, 2010, under the functional category of “Esters.” Other esters suitable for use in the personal care composition include those known as polyhydric alcohol esters and glycerides.

Other suitable oils include amides (e.g., compounds having an amide functional group while being liquid at 25° C. and insoluble in water). Suitable amides include N-acetyl-N-butylaminopropionate, isopropyl N-lauroylsarcosinate, and N,N,-diethyltoluamide and those disclosed in U.S. Pat. No. 6,872,401.

Other suitable oils include ethers. Suitable ethers include saturated and unsaturated fatty ethers of a polyhydric alcohol, and alkoxylated derivatives thereof. Exemplary ethers include C₄₋₂₀ alkyl ethers of polypropylene glycols, and di-C₈₋₃₀ alkyl ethers. Suitable examples of these materials include PPG-14 butyl ether, PPG-15 stearyl ether, dioctyl ether, dodecyl octyl ether, and mixtures thereof.

The skin care composition may include an emulsifier. An emulsifier may be desirable when the composition is provided in the form of an emulsion or if immiscible materials are being combined. The cosmetic compositions herein may include from 0.05%, 0.1%, 0.2%, 0.3%, 0.5%, or 1% to 20%, 10%, 5%, 3%, 2%, or 1% emulsifier. Emulsifiers may be nonionic, anionic or cationic. Non-limiting examples of emulsifiers are disclosed in U.S. Pat. No. 3,755,560, U.S. Pat. No. 4,421,769, and McCutcheon's, Emulsifiers and Detergents, 2010 Annual Ed., published by M. C. Publishing Co. Other suitable emulsifiers are further described in the Personal Care Product Council's International Cosmetic Ingredient Dictionary and Handbook, Thirteenth Edition, 2006, under the functional category of “Surfactants—Emulsifying Agents.”

Suitable emulsifiers include the following classes of ethers and esters: ethers of polyglycols and of fatty alcohols, esters of polyglycols and of fatty acids, ethers of polyglycols and of fatty alcohols which are glycosylated, esters of polyglycols and of fatty acids which are glycosylated, ethers of C₁₂₋₃₀ alcohols and of glycerol or of polyglycerol, esters of C₁₂₋₃₀ fatty acids and of glycerol or of polyglycerol, ethers of oxyalkylene-modified C₁₂₋₃₀ alcohols and of glycerol or polyglycerol, ethers of C₁₂₋₃₀ fatty alcohols comprising and of sucrose or of glucose, esters of sucrose and of C₁₂₋₃₀ fatty acids, esters of pentaerythritol and of C₁₂₋₃₀ fatty acids, esters of sorbitol and/or of sorbitan and of C₁₂₋₃₀ fatty acids, ethers of sorbitol and/or of sorbitan and of alkoxylated sorbitan, ethers of polyglycols and of cholesterol, esters of C₁₂₋₃₀ fatty acids and of alkoxylated ethers of sorbitol and/or sorbitan, and combinations thereof.

Linear or branched type silicone emulsifiers may also be used. Particularly useful polyether modified silicones include KF-6011, KF-6012, KF-6013, KF-6015, KF-6015, KF-6017, KF-6043, KF-6028, and KF-6038 from Shin Etsu. Also particularly useful are the polyglycerolated linear or branched siloxane emulsifiers including KF-6100, KF-6104, and KF-6105 from Shin Etsu.

Emulsifiers also include emulsifying silicone elastomers. Suitable emulsifying silicone elastomers may include at least one polyalkyl ether or polyglycerolated unit. Polyoxyalylenated emulsifying silicone elastomers that may be used in at least one embodiment of the invention include those sold by Shin-Etsu Silicones under the names KSG-21, KSG-20, KSG-30, KSG-31, KSG-32, KSG-33; KSG-210 (dimethicone/PEG-10/15 crosspolymer dispersed in dimethicone); KSG-310 (PEG-15 lauryl dimethicone crosspolymer); KSG-320 (PEG-15 lauryl dimethicone crosspolymer dispersed in isododecane); KSG-330 (PEG-15 lauryl dimethicone crosspolymer dispersed in triethylhexanoin), KSG-340 (PEG-10 lauryl dimethicone crosspolymer and PEG-15 lauryl dimethicone crosspolymer). Other silicone emulsifying elastomers are supplied by Dow Corning™, including PEG-12 dimethicone crosspolymers (DC 9010 and 9011). Other suitable silicone emulsifiers sold by Dow Corning include DC9010 and DC9011. Polyglycerolated emulsifying silicone elastomers are disclosed in PCT/WO 2004/024798. Such elastomers include Shin-Etsu's KSG series, such as KSG-710 (dimethicone/polyglycerin-3 crosspolymer dispersed in dimethicone); or lauryl dimethicone/polyglycerin-3 crosspolymer dispersed in a variety of solvent such as isododecane, dimethicone, triethylhexanoin, available as KSG-810, KSG-820, KSG-830, or KSG-840 from Shin-Etsu.

Structuring agents may be used to increase viscosity, thicken, solidify, or provide solid or crystalline structure to the skin care composition. Structuring agents are typically grouped based on solubility, dispersibility, or phase compatibility. Examples of aqueous or water structuring agents include polymeric agents, natural or synthetic gums, polysaccharides, and the like. Other exemplary classes of polymeric structuring agents include but are not limited to carboxylic acid polymers, polyacrylamide polymers, sulfonated polymers, high molecular weight polyalkylglycols or polyglycerins, copolymers thereof, hydrophobically modified derivatives thereof, and mixtures thereof. In certain embodiments, the composition may comprises from about 0.0001%, 0.001%, 0.01%, 0.05%, 0.1%, 0.5%, 1%, 2%, 3%, 5% to about 25%, 20%, 10%, 7%, 5%, 4%, or 2%, by weight of the composition, of one or more structuring agents.

Examples of oil structuring agents include silicone and organic based materials. Suitable ranges of oil structuring agents are from 0.01%, 0.05%, 0.1%, 0.5%, 1%, 2.5%, 5%, or 10% to 30%, 25%, 20%, 15%, 10%, or 5%. Suitable oil phase structuring agents may be silicone based, such as silicone elastomers, silicone gums, silicone waxes, and linear silicones polymers which have a degree of polymerization that allows the silicone to increase the viscosity of the oil phase.

Suitable silicone elastomers may be in the powder form, or dispersed or solubilized in solvents such as volatile or nonvolatile silicones, or silicone compatible vehicles such as paraffinic hydrocarbons or esters. Examples of silicone elastomer powders include vinyl dimethicone/methicone silsesquioxane crosspolymers like KSP-100, KSP-101, KSP-102, KSP-103, KSP-104, KSP-105, available from Shin-Etsu, hybrid silicone powders that contain a fluoroalkyl group like KSP-200, available from Shin-Etsu, which is a fluoro-silicone elastomer, and hybrid silicone powders that contain a phenyl group such as KSP-300, available from Shin-Etsu, which is a phenyl substituted silicone elastomer; and DC 9506 available from Dow Corning. Examples of silicone elastomer dispersions include dimethicone/vinyl dimethicone crosspolymers supplied by a variety of suppliers including Dow Corning Corporation under the tradenames DC9040 or DC9041, Momentive under the tradename SFE 839, or Shin-Etsu Silicones under the tradenames KSG-15, 16, 18. KSG-15 has the INCI name cyclopentasiloxane (and) dimethicone/vinyl dimethicone crosspolymer. KSG-18 has the INCI name diphenylsiloxy phenyl trimethicone (and) dimethicone/phenyl vinyl dimethicone crossoplymer. Silicone elastomers may also be purchased from Grant Industries under the Gransil trademark. Other suitable silicone elastomers have long chain alkyl substitutions such as lauryl dimethicone/vinyl dimethicone crosspolymers supplied by Shin Etsu under the tradenames KSG-41, KSG-42, KSG-43, and KSG-44, wherein the elastomer is dispersed in solvents including mineral oil, isodocane, triethylhexanoin, or squalene, respectively. Other suitable silicone elastomers may have polyglycerine substitutions such as lauryl dimethicone/polyglycerin-3 crosspolymers supplied by Shin Etsu under the tradenames KSG-810, KSG-820, KSG-830, and KSG-840, wherein the elastomer is dispersed in solvents including mineral oil, isodocane, triethylhexanoin, or squalene, respectively. Other suitable silicone elastomers may have polyglycol substitutions such as PEG-15/lauryl dimethiconecrosspolymers supplied by Shin Etsu under the tradenames KSG-310, KSG-320, KSG-330, and KSG-340, wherein the elastomer is dispersed in solvents including mineral oil, isodocane, triethylhexanoin, or squalene, respectively. Other suitable silicone elastomers having polyglycol substitutions include Shin Etsu's KSG-210, a dimethicone/PEG-10/15 crosspolymer in dimethicone.

Silicone gums are another oil phase structuring agent. Silicone gums suitable for use herein may have a viscosity ranging from 500,000 to 100 million cSt at 25° C., from 600,000 to 20 million cSt, from about 600,000 to 12 million cSt. Suitable silicone gums include those sold by Wacker-Belsil under the trade names CM3092, Wacker-Belsil 1000, or Wacker-Belsil DM 3096. A particularly suitable silicone gum is as dimethiconol, available from Dow Corning Corporation under the trade name 1-1254 Fluid, 2-9023 Fluid, and 2-9026 Fluid. Dimethiconol is often sold as a mixture with a volatile or nonvolatile silicone such as Dow Corning 1401 Fluid, 1403 Fluid, and 1501 Fluid.

Another type of oily phase structuring agent includes silicone waxes. Silicone waxes may be referred to as alkyl silicone waxes and are semi-solids or solids at room temperature. The term “alkyl silicone wax” means a polydimethylsiloxane having a substituted long chain alkyl (e.g., C₁₆ to C₃₀) that confers a semi-solid or solid property to the siloxane. Examples of such silicone waxes include stearyl dimethicone, which may be purchased from Evonik Goldschmidt GmbH under the tradename Abil Wax 9800 or from Dow Corning under the tradename 2503. Another example is bis-stearyl dimethicone (which may be purchased from Gransil Industries under the tradename Gransil A-18), behenyl dimethicone, or behenoxy dimethicone. Suitable silicone waxes are disclosed in U.S. Pat. Nos. 5,413,781 and 5,725,845, and further include alkylmethyl polysiloxanes, C₁₀₋₆₀ alkyl dimethicones, and mixtures thereof.

Other non-limiting examples of oil phase structuring agents include natural and synthetic waxes (e.g., natural animal, vegetable and mineral waxes and synthetic waxes made therefrom). Still other examples of structuring agents include natural or synthetic montmorillonite minerals, silicas, silicates, silica silylate, and alkali metal or alkaline earth metal derivatives thereof.

Optional Ingredients

The cosmetic compositions herein may optionally include ingredients useful for regulating and/or improving a condition of mammalian skin. Some non-limiting examples of such optional ingredients include vitamins; peptides and peptide derivatives; sugar amines, sunscreen actives (or sunscreen agents) and/or ultraviolet light absorbers, phytosterols, salicylic acid compounds, hexamidines, dialkanoyl hydroxyproline compounds, flavonoids, retinoid compounds, botanicals, N-acyl amino acid compounds, their derivatives, and combinations thereof.

The present cosmetic compositions may include a sugar amine, which is also known as an amino sugar. Exemplary sugar amines suitable for use herein are described in PCT Publication No. WO 02/076423 and U.S. Pat. No. 6,159,485. The sugar amine may be present at an amount of from 0.01% to 15%, from 0.1% to 10%, or from 0.5% to 5% by weight based on the weight of the cosmetic composition. Sugar amines can be synthetic or natural in origin and can be used as pure compounds or mixtures of compounds (e.g., extracts from natural sources or mixtures of synthetic materials). A particularly suitable example of a sugar amine is glucosamine and its salts, which may be found in certain shellfish or derived from fungal sources. Other examples of sugar amines include N-acetyl glucosamine, mannosamine, N-acetyl mannosamine, galactosamine, N-acetyl galactosamine, their isomers (e.g., stereoisomers), and their salts (e.g., HCl salt).

The present cosmetic compositions may include a vitamin B₃ compound (e.g., niacinamide). Vitamin B₃ compounds may regulate skin conditions as described in U.S. Pat. No. 5,939,082. The cosmetic composition may contain from 0.001% to 50%, from 0.01% to 20%, from 0.05% to 10%, from 0.1% to 7%, or even from 0.5% to 5%, by weight based on the weight of the cosmetic composition. Some exemplary derivatives of the foregoing vitamin B₃ compounds include nicotinic acid esters, including non-vasodilating esters of nicotinic acid (e.g., tocopheryl nicotinate, myristyl nicotinate). Examples of suitable vitamin B₃ compounds are commercially available from a number of sources (e.g., the Sigma Chemical Company, ICN Biomedicals, Inc., and Aldrich Chemical Company).

The present cosmetic compositions may include a salicylic acid compound, its esters, its salts, or combinations thereof. The salicylic acid compound may include from 0.0001% to 25%, from 0.001% to 15%, from 0.01% to 10%, from 0.1% to 5%, or even from 0.2% to 2%, by weight based on the weight of the cosmetic composition.

The present cosmetic compositions may include hexamidine compounds, its salts and derivatives. The hexamidine may be present at an amount of from 0.0001% to 25%, or from 0.001% to 10%, or from 0.01% to 5%, or from 0.02% to 2.5% by weight based on the weight of the composition. As used herein, hexamidine derivatives include any isomers and tautomers of hexamidine compounds including, but not limited to, organic acids and mineral acids, for example sulfonic acid, carboxylic acid, etc. The hexamidine compounds include hexamidine diisethionate, commercially available as Eleastab® HP100 from Laboratoires Serobiologiques.

The present cosmetic compositions may include a flavonoid compound. Flavonoids are broadly disclosed in U.S. Pat. Nos. 5,686,082 and 5,686,367. Examples of some flavonoids are one or more flavones, one or more isoflavones, one or more coumarins, one or more chromones, one or more dicoumarols, one or more chromanones, one or more chromanols, isomers (e.g., cis/trans isomers) thereof, and mixtures thereof. Some examples include flavones and isoflavones, such as daidzein (7,4′-dihydroxy isoflavone), genistein (5,7,4′-trihydroxy isoflavone), equol (7,4′-dihydroxy isoflavan), 5,7-dihydroxy-4′-methoxy isoflavone, soy isoflavones (a mixture extracted from soy), and mixtures thereof. Flavonoid compounds useful herein are commercially available from a number of sources, e.g., Indofine Chemical Company, Inc., Steraloids, Inc., and Aldrich Chemical Company, Inc. The flavonoid compounds may comprise from 0.01% to 20%, from 0.1% to 10%, or from 0.5% to 5%, by weight based on the weight of the cosmetic composition.

The present cosmetic compositions may comprise one or more N-acyl amino acid compounds. The amino acid can be one of any of the amino acids known in the art. A list of possible side chains of amino acids known in the art are described in Stryer, Biochemistry, 1981, published by W.H. Freeman and Company. R¹ can be C₁ to C₃₀, saturated or unsaturated, straight or branched, substituted or unsubstituted alkyls; substituted or unsubstituted aromatic groups; or mixtures thereof. The N-acyl amino acid compound may be selected from the group consisting of N-acyl Phenylalanine, N-acyl Tyrosine, their isomers, their salts, and derivatives thereof. The amino acid can be the D or L isomer or a mixture thereof. One example of an amino acid derivative is N-undecylenoyl-L-phenylalanine, which belongs to the class of N-acyl phenylalanine amino acid derivatives. This exemplary amino acid derivative includes an acyl group which is a C₁₁ mono-unsaturated fatty acid moiety and the L-isomer of phenylalanine. One example of N-undecylenoyl-L-phenylalanine is commercially available under the tradename Sepiwhite® from SEPPIC. The N-acyl amino acid derivative may be present at an amount of from 0.0001% to 25%, from 0.001% to 10%, from 0.01% to 5%, or from 0.02% to 2.5% by weight of the cosmetic composition.

The present cosmetic compositions may include a retinoid, which may be present at an amount of from 0.001% to 10%, from 0.005% to 2%, from 0.008% to 1%, or from 0.01% to 0.5% by weight based on the weight of the composition. “Retinoid” as used herein means natural and synthetic analogs of Vitamin A, or retinol-like compounds which possess the biological activity of Vitamin A in the skin, as well as the geometric isomers and stereoisomers of these compounds. The retinoid may be selected from retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), or mixtures thereof.

The present cosmetic compositions may contain a peptide, including but not limited to, di-, tri-, tetra-, penta-, and hexa-peptides and derivatives thereof. The cosmetic compositions may contain from 1×10⁻⁷% to 20%, or from 1×10⁻⁶% to 0%, or from 1×10⁻⁵% to 5% by weight of the composition or a peptide. Peptides may contain ten or fewer amino acids and their derivatives, isomers, and complexes with other species such as metal ions (e.g., copper, zinc, manganese, magnesium, and the like). Peptide refers to both naturally occurring and synthesized peptides. Also useful herein are naturally occurring and commercially available compositions that contain peptides. Some examples of peptides include the dipeptide carnosine (beta-ala-his), the tripeptide gly-his-lys, the pentapeptide lys-thr-thr-lys-ser, lipophilic derivatives of peptides, and metal complexes of the above, e.g., copper complex of the tripeptide his-gly-gly (also known as lamin). A commercially available tripeptide derivative-containing composition is Biopeptide CL®, which contains 100 ppm of palmitoyl-gly-his-lys, is commercially available from Sederma. A preferred commercially available pentapeptide derivative-containing composition is Matrixyl®, which contains 100 ppm of palmnitoyl-lys-thr-thr-lys-ser is commercially available from Sederma.

The present cosmetic compositions may contain one or more water-soluble vitamins. Examples of water-soluble vitamins including, but are not limited to, water-soluble versions of vitamin B, vitamin B derivatives, vitamin C, vitamin C derivatives, vitamin K, vitamin K derivatives, vitamin D, vitamin D derivatives, vitamin E, vitamin E derivatives, provitamins thereof, such as panthenol and mixtures thereof. The cosmetic compositions may contain from 0.0001% to 50%, or from 0.001% to 10%, from 0.01% to 8%, or from 0.1% to 5% by weight based on the weight of the composition.

The present cosmetic compositions may contain a sunscreen active. Sunscreen actives include both sunscreen agents and physical sunblocks. Sunscreen actives may be organic or inorganic. A wide variety of conventional sunscreen actives may be used. Sagarin, et al., at Chapter VIII, pages 189 et seq., of Cosmetics Science and Technology (1972), discloses numerous suitable actives. Some non-limiting examples of sunscreens include 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, titanium dioxide, and mixtures of these compounds. Some organic sunscreen actives are 2-ethylhexyl-p-methoxycinnamate, butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, zinc oxide, titanium dioxide, and mixtures thereof. The sunscreen active may be present at an amount of from 1% to 20%, or from 2% to 10% by weight based on the weight of the composition. Exact amounts may vary depending upon the sunscreen chosen and the desired Sun Protection Factor (SPF).

The present cosmetic compositions may contain a conditioning agent such as a humectant, a moisturizer, or a skin conditioner. A variety of these materials can be employed and each may be present at a level of from 0.01% to 20%, from 0.1% to 10%, from 0.5% to 7% by weight based on the weight of the composition. Some non-limiting examples of conditioning agents include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fucose); hyaluronic acid; lactamide monoethanolamine; acetamide monoethanolamine; panthenol; allantoin; and mixtures thereof. Also useful herein are the propoxylated glycerols described in U.S. Pat. No. 4,976,953. Also useful are various C₁-C₃₀ monoesters and polyesters of sugars and related materials. These esters are derived from a sugar or polyol moiety and one or more carboxylic acid moieties.

The present cosmetic compositions may include other optional ingredients such as one or more colorants (pigments, dyes, lakes, combinations of these and the like), surfactants and/or film-forming compositions. The present cosmetic compositions may be in any one of a variety of forms known in the art, including, for example, an emulsion, lotion, milk, liquid, solid, cream, gel, mouse, ointment, paste, serum, stick, spray, tonic, aerosol, foam, pencil, and the like. The cosmetic compositions may also be incorporated into shave prep products, including, for example, gels, foams, lotions, and creams, and include both aerosol and non-aerosol versions. Other cosmetic compositions include antiperspirant, deodorant, and personal cleaning compositions such as soap and shampoo. Suitable examples of cosmetic compositions are disclosed in U.S. Pub. No. 2009/0017080 filed by Tanner, et al., on Mar. 13, 2008; U.S. Pub. No. 2010/0112100 filed by Willemin, et al., on Jan. 11, 2010; PCT Pub. No. WO2010/129313 filed by Susak, et al., on Apr. 28, 2010; U.S. Pub. No. 2011/0280647 filed by Wilson, et al., on Feb. 14, 2011; U.S. Pub. No. 20050244442 filed by Sabino, et al., on Apr. 28, 2005; European Pub. No. EP2025364 filed by Alberius, et al., on Aug. 13, 2007; and U.S. Pat. Nos. 6,017,552, 6,060,547, 7,022,346, 7,404,966, 7,772,214 and 7,871,633.

The present cosmetic compositions may be prepared according to conventional methods known in the art for making such compositions. Such methods may include mixing ingredients in one or more steps to achieve a relatively uniform state, with or without heating, cooling, application of vacuum, and the like. For example, emulsions may be prepared by first mixing the aqueous phase materials separately from the fatty phase materials and then combining the two phases as appropriate to yield the desired continuous phase. In certain embodiments, the compositions may be prepared to provide suitable stability (physical stability, chemical stability, photostability, etc.) and/or delivery of active materials. The composition may be provided in a package sized to store a sufficient amount of the composition for a treatment period. The size, shape, and design of the package may vary widely. Some package examples are described in U.S. Pat. Nos. D570,707; D391,162; D516,436; D535,191; D542,660; D547,193; D547,661; D558,591; D563,221; and U.S. Publication Nos. 2009/0017080; 2007/0205226; and 2007/0040306.

Method of Use

The cosmetic compositions disclosed herein may be suitable for use as topical skin care or color cosmetic products, which may be applied as part of a user's routine makeup or personal care regimen. Additionally or alternatively, the cosmetic compositions herein may be used on an “as needed” basis. In certain embodiments, a skin care product such as a moisturizing cream, lotion or ointment that includes a cosmetically acceptable carrier and an effective amount of a skin commensal prebiotic agent may be topically applied to one or more target areas of a user's skin (e.g., face, forearms, hands or portions of these) to provide a skin care benefit or otherwise improve the health and/or appearance of the skin in the target area(s). In certain embodiments, the skin commensal prebiotic agent may be incorporated into a color cosmetic product such as a foundation that is applied to a user's face or portions thereof as part of a daily beauty regimen.

In certain embodiments, particular areas of the skin may be identified as being in need of a skin care benefit that can be addressed through the use of the cosmetic compositions herein. For example, areas of the face (e.g., nose, cheeks, forehead, chin, around the eyes), the front and back of the neck, the top of a hand, the top of a forearm, the shoulders and/or a major body fold may be identified as being in need of treatment by the present prebiotic, topical cosmetic compositions. Of course, it is to be appreciated that the cosmetic compositions disclosed herein may be applied to any portion of skin on the body (e.g., feet, legs, back, upper arm, torso, buttocks) to provide a cosmetic benefit, and such portions of the skin may be identified as target areas.

In certain embodiments, the topical cosmetic compositions herein may be used in conjunction with a probiotic or probiotic-derived substance (e.g., probiotic lysate), which may be provided in the form of a topical composition and/or an orally ingestible composition. In certain embodiments, the topical cosmetic compositions herein may be used in conjunction with an orally ingestable prebiotic (e.g., GOS), probiotic (e.g., Bifido bacteria) and/or nutritional supplement (e.g., omega-3 fatty acid). For example, the present topical cosmetic compositions may be marketed in a kit that also includes an orally ingestible GI prebiotic, probiotic, and/or probiotic derived compound. In certain embodiments, the kit may include a first topical composition incorporating an effective amount of a first skin commensal prebiotic such as GOS and a second topical composition incorporating a probiotic, probiotic lysate and/or a GI or second skin commensal prebiotic. GI prebiotics are generally recognized as being 1) resistant to degradation by stomach acid, mammalian enzymes and hydrolysis; 2) fermentable by at least one type (e.g., genus or species) of desirable GI microorganism; and 3) capable of selectively stimulating growth and/or activity of at least one type of desirable GI microorganism. Several non-limiting examples of GI prebiotic agents are shown in FIG. 8.

The topical cosmetic compositions herein may also include a probiotic or probiotic-derived substance such as a lysate that provides a skin care benefit in combination with a skin commensal prebiotic. The probiotic may be a skin commensal microorganism or a GI microorganism or a lysate obtained from one of these. For example, the cosmetic compositions herein may include one or members of the Bifidobacterium genus, Lactobacillus genus, Enterococcus genus, Streptococcus genus or Staphylococcus genus; Leuconostoc mesenteroides subsp dextranicum; Pediococcus acidilactici; Sporolactobacillus inulinus; Streptococcus salvarius subsp. thermophilus; Saccharomyces (cerevisiae or else boulardii); Bacillus (cereus var toyo or subtilis); Bacillus coagulans; Bacillus licheniformis; Escherichia coli strain nissle; Propionibacterium freudenreichii; and mixtures of these. Nonlimiting examples of GI probiotic microorganisms and probiotic lysates are disclosed in U.S. Pub. No. 20100203094 filed by Amar, et al., on Jan. 12, 2010; U.S. Pub. No. 20100226892 filed by Gueniche on Mar. 4, 2010; PCT Pub. No. WO 2011/048554 filed by Breton on Oct. 20, 2010; and PCT Pub. Nos. WO 2011/070508 and WO 2011/070509 both filed by Gueniche, et al., on Dec. 7, 2010.

The present cosemtic compositions may be applied one or more times per day as part of a user's regular beauty regimen (e.g., showering, applying makeup, applying moisturizers or other skin care or hair care products). The present topical cosmetic compositions may be applied more than once per day, for example, once at the beginning of the day, once in the middle of the day, and/or once at the end of the day. In some instances, the present cosmetic compositions may be applied whenever a user applies or reapplies other cosmetic compositions such as lipstick or mascara. In some instances, it may be desirable to apply the present cosmetic compositions every other day, two or three times per week, weekly, biweekly or monthly, as desired. It may be desirable to apply the present cosmetic compositions such that at least a portion of the composition (e.g., the prebiotic portion) is present on the user's skin for at least an hour (e.g, from 1 to 24 hours, from 2 to 20 hours, from 4 to 16 hours, or from 8-12 hours). In certain embodiments, it may be desirable to apply the composition such that at least a portion of the composition is present on skin for more than a day (e.g., 1-7 days, 2-6 days, 3-5 days, or even 4 days). In certain embodiments, it may be desirable to apply the present cosmetic compositions at one or more of the foregoing frequencies for at least two consecutive or nonconsecutive application periods. For example, the composition may be applied once per day for 2, 3, 4, 5, 6, or 7 consecutive or nonconsecutive days. In another example, the present cosmetic composition may be applied every other day for a month or more. Additionally or alternatively, the present cosmetic compositions may be used in conjunction with an orally ingested probiotic, probiotic derived composition (e.g., lysate) and/or prebiotic in one or more of the foregoing periods of time.

### Test Methods

Preparing Starter Cultures, Work Cultures and Test Samples

Obtain a test specimen for each of C. jeikeium, S. epidermidis, and P. acnes from a suitable source. A particularly suitable source is American Type Culture Collection (ATCC) in Manassas, Va. as Catalog Nos. 43734, 12228, and 11827, respectively. The microbes are each grown in a starter culture using sterile media, which may be sterilized using conventional methods (e.g., autoclave). S. epidermidis is grown in a starter culture of brain heart infusion media (“BHI”); C. jeikeium is grown in a starter culture of BHI media supplemented with 0.1% Tween 80 (“BHIT”); and P. acnes is grown in a starter culture of reinforced clostridial broth (“RCB”). The BHI media is made by adding 37 grams of a commercially available powder of peptic digest of animal tissue, sodium chloride, dextrose, pancreatic digest of gelatin, and disodium phosphate to 1 liter of USP water. The RCB is made by adding 38 grams of a commercially available powder of casein enzymatic hydrolysate, beef and yeast extract, dextrose, sodium chloride, sodium acetate, starch, and 1-cysteine hydrochloride to 1 liter of USP water. Glycerol stock inoculums of each of the three kinds of bacteria are prepared by mixing 0.75 ml of a log culture with 0.25 ml of 80% glycerol and storing at −80° C. until use.

On day 1, the starter culture of BHIT is made by inoculating the BHIT media in a 50:1 ratio with C. jeikeium in a suitable vessel (i.e., 1 ml glycerol stock inoculum to 50 ml BHIT media). Also on day 1, the starter culture of RCB is made by inoculating the RCB media in a 50:1 ratio with P. acnes in a suitable vessel (i.e., 1 ml glycerol stock inoculum to 50 ml RCB media). The starter culture containing C. jeikeium is incubated aerobically at 33-37° C. for 46 to 48 hours. The starter culture containing P. acnes is incubated anaerobically at 35-37° C. for 46 to 48 hours.

On day 2, the starter culture of BHI is made by inoculating the BHI media in a 50:1 ratio with S. epidermidis in a suitable vessel (i.e. 1 ml glycerol stock inoculum to 50 ml BHI media) followed by aerobic incubation at 33-37° C. for 22 to 26 hours.

On day 3, the three starter cultures are harvested by room-temperature centrifugation at a speed sufficient to pelletize the bacteria but maintain viability (e.g., 8500 rpm in a Sorvall Evolution RC centrifuge. The bacterial pellets from the starter cultures are washed in a 0.90% w/v saline solution (“normal saline”), re-pelleted, and re-suspended in enough normal saline to provide a work culture with a bacterial concentration of between 0.5×10⁷ CFU/ml to 5×10⁸ CFU/ml.

0.05%, 0.5% and 1% test samples may be prepared as follows. However, it is to be appreciated that the following method may be modified, as is commonly known in the art, to provide a test sample with the desired final volume or concentration.

A 10× working stock solution of the test agent may be prepared by adding 1 g of dry irradiated test material (e.g., GOS) to 10 ml of water (10% w/v) and filtering the solution through a 0.2 micron filter unit.

A 0.05% test sample may be provided by adding 0.5 ml of the 10× working stock solution to 9.5 ml water to give a 0.5% diluted working stock solution. 0.1 ml of this diluted working stock solution may then be combined with 0.8 ml of minimal carbon medium and 0.1 ml of the desired work culture to provide a final volume of 1 ml in a suitable test vessel (e.g., in each well of a 96-well, deep-well plate or a flask).

A 0.5% test sample may be provided by adding 5 ml of the 10× working stock solution to 5 ml of water to make a diluted working stock solution, and then combining 0.1 ml of the diluted working stock solution with 0.8 ml of minimal carbon medium and 0.1 ml of the desired work culture to provide a final volume of 1 ml in a suitable test vessel.

A 1% test sample may be provided by combining 0.1 ml of the 10× working stock solution with 0.8 ml of minimal carbon medium and 0.1 ml of the desired work culture to provide a final volume of 1 ml in a suitable test vessel.

A water control is provided by replacing the test material with water. The time at which the test materials are added to the reaction vessel to form the test sample is T=0. All transfers of media or other ingredients may be performed, for example, by using an Eppendorf Research Series Adjustable Volume Pippetter with a suitable volume range (e.g., 100 μl to 1000 μl or 2 μl to 20 μl), available from Fisher Scientific, Pittsburgh, Pa.

Prior to sampling a well, the contents of each well are mixed by pipetting up and down the well, which is a conventional mixing technique known in the art.

ATP Test

The ATP Test may be used to determine the level of adenosine triphosphate present in a test sample. To measure the ATP in each well, a sample (e.g., 100 microliters) is removed from each well of the reaction vessel using a suitable transfer apparatus and placed in a 96-well, black well plate. Optionally, enough glucose may be added to the wells containing S. epidermidis to reach a final concentration of 1% v/v and waiting at least 5 minutes at room temperature. It is believed, without being limited by theory, that S. epidermidis tends to use up its ATP faster than the other two microorganisms when stressed (i.e., starved). Thus, adding glucose may “prime” the S. epidermidis and provide a baseline ATP level that is commensurate with a corresponding plate count value. However, it may be desirable to refrain from adding glucose to the wells containing the S. epidermidis in order to potentially increase the dynamic range for measurable prebiotic activity. After placing the test samples in the black-well plate, the ATP level of the test sample is measured by adding an equal volume of ATP reagent (e.g., BacTiter Glo, from Promega Corporation) to each well. For example, a 100 ul sample would get 100 ul of ATP reagent according to the manufacturer instructions. The plates are then incubated at room temperature for fifteen minutes with shaking at 750 rpm. The luminescence of the cultures may be measured with a suitable luminescent plate reader such as, for example, a Victor X Multi Label brand plate reader available from Wallac/PerkinElmer in Waltham, Mass. The measured luminescence is recorded as an ATP value. The reaction vessels are sampled at T=0, T=24 hours and T=48 hours. The ATP level measured at T=0 is measured as soon as possible after making the test samples, and in no event longer than 30 minutes. Run the test three times for each sample and average the results to provide an ATP value.

Plate Count Test

The Plate Count test may be used for bacterial count assessments. To begin, remove 10 μl of test sample from each triplicate vessel at T=0, for a total of 30 ul, and place it in 970 ul of normal saline. Serially dilute samples as needed to allow a countable range of 20-300 colonies per plate (e.g., 1:10 to 1:10,000), plate the samples in duplicate on a suitable medium for each organism tested (e.g., Brucella Blood Agar (“BBA”) TSA, TSA-0.1% Tween, RCA) by placing 50 ul of the appropriate dilutions on each plate with a suitable plating technique as is commonly known to those skilled in the art. Incubate the resultant plates at 33-37° C. in the presence of oxygen or 35-37° C. anaerobically (depending on whether the microorganism prefers aerobic or anaerobic conditions) and analyze 48 to 72 hours later using conventional colony counting techniques known in the art to determine the number of colony forming units. Average the values of the duplicate plates to provide a bacterial count value.

In Vivo Study

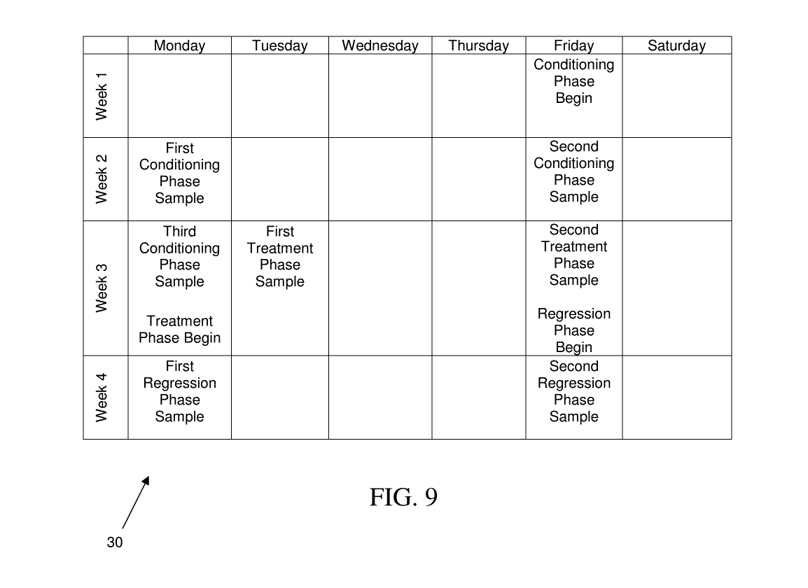

An in vivo study may be conducted to confirm the prebiotic potential of a test agent that was predicted in vitro. In this study protocol, 24 female volunteers are selected to be test subjects. The test subjects must meet the following inclusion criteria and must not meet any of the following exclusion criteria.
Inclusion Criteria
   1. Female
   2. Age 18 to 65 years
   3. Self-reported good general health
   4. Forearm supports the template
Exclusion Criteria
   1. Antibiotic use in the last 2 weeks (or during study)
   2. Known food allergies to milk or beets
   3. Inflammation, visible cuts, abrasions, etc in the sample area
   4. Persistent skin condition, such as eczema, causing recurring skin rashes, dryness or itching
Subject Instructions/Restrictions. The test subjects agreed to observe the following instructions/restrictions.
   1. Abstain from using any other product on their forearms other than those supplied for the duration of the study (including, for example, moisturizing lotions and sunscreen)
   2. Use caution when washing hands. Do not allow soap to contact test areas on forearm (however, it is recognized that some incidental soap contact may be unavoidable during showering)
   3. Use only the supplied products for the duration of the study including the 10 day conditioning period and 8 day regression period:
      a. Olay Ultra Moisture With Shea Butter brand bar soap (it is important NOT to use antibacterial soap)
      b. Pantene brand Shampoo (it is important NOT to use anti dandruff shampoo)
      c. Pantene brand Conditioner (if desired)
   4. On all sampling and treatment days (see study calendar in FIG. 9), abstain from forearm washing. Showering is permitted; however, DO NOT physically wash forearms.
   5. During the Treatment Phase (see study calendar in FIG. 9), abstain from wearing clothing with long sleeves (i.e., clothing that covers the forearm).
   6. Abstain from bathing (soaking/being submerged in water) throughout study including Conditioning and Regression Phases
   7. Abstain from swimming or sitting in chlorinated water for the duration of the study
   8. Abstain from excessive sun exposure (artificial or natural sun light)
   9. Inform the study investigators if a change in health status is experienced during the study
   10. Do not participate in any other studies involving the forearm while participating in this study

Study Design

The study includes three phases. The first phase is the Conditioning Phase, during which a baseline level of bacterial counts is obtained from each test subject at the target sites on the forearm. The second phase is the Treatment Phase, during which the target sites on the forearm are exposed to the test agent (e.g., GOS) and samples are taken to determine whether a change in the bacterial counts has occurred relative to the baseline. The third phase is the Regression Phase, during which the target areas on the forearm are no longer exposed to the test agent, and samples are taken to determine whether a change in bacterial counts has occurred relative to the Treatment Phase and/or the Conditioning Phase. A chart 30 is provided in FIG. 9 to show the timeline for the phases of the study as well as when sampling occurs.

Conditioning Phase.

As illustrated in the chart 30 of FIG. 9, the Conditioning Phase begins on Friday of week 1. The test subjects are given instructions and personal cleansing products to be used during the study (i.e., shampoo, conditioner and bar soap). The test subject are instructed to use only the provided products for all showering and follow their typical habits and practices as it pertains to showering except on the three sampling mornings (i.e., Monday and Friday of week 2 and Monday of week 3). The test subjects are instructed to report to the study location on Monday and Friday of week 2 and Monday of week 3 for forearm microbial sampling, which is described in more detail below. On the three sampling mornings, the test subjects do not wash their forearms (no soap or physical scrubbing) prior to sampling.

Treatment Phase

The Treatment Phase begins on Monday of week 3. The test subjects report to the study location each morning Monday to Thursday of week 3 between 7:30 and 9:30 AM, and return each afternoon between 1:00 and 3:00 PM for application of the test material on the prescribed forearm sites. The test subjects do not wash their forearms (no soap or physical scrubbing) or wear any covering over their forearms throughout the Treatment Phase. Rinsing the forearm with warm water is permitted after sampling (when applicable) and before treatment. During the Treatment Phase, forearm microbial samples are collected in the morning on Monday (third Conditioning Phase sample), Tuesday (first Treatment Phase sample) and Friday (second Treatment Phase sample).

Regression Phase

The Regression Phase begins on Friday of week 3. The test subjects report to the study site on Monday of week 4 for forearm microbial sampling during regression. The test subjects follow their typical habits and practices as it pertains to showering except on the sampling morning. On the sampling morning, subjects do not wash their forearms (no soap or physical scrubbing) prior to sampling.

Sampling and Treatment

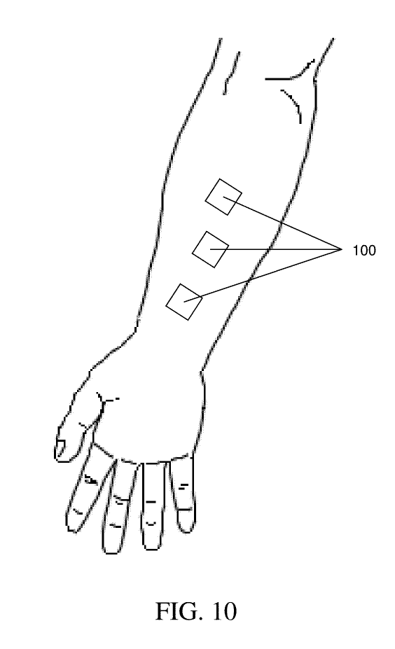

The forearm of each test subject is marked using a fixed template with a sufficient number of 1.5 inch×1.5 inch square areas 100, as illustrated in FIG. 10. The squares each identify a target test area on the forearm. In the example illustrated in FIG. 10, six test agents may be tested (i.e., three on each arm) or three test agents may be tested twice (i.e., duplicated on each forearm), or any combination of these. On the other hand, if there is only one test agent, then two squares 100 on each forearm may be sufficient to provide suitable test areas for a test agent and a control (e.g., a water control). If, during the course of the study, the markings fade or otherwise become hard to see, the corners of each square 100 may be identified with a suitable marking device (e.g., permanent marker) to permit consistent sampling and treatment. The test agent(s) used for the treatments is provided in the form of an aqueous test solution. After preparation, the test solution is filtered (0.2 um) under aseptic conditions and then transferred to individual sterile vials (1 mL) for daily use per test subject. Fifty microliters (μL) are applied to each target area on the forearm during each visit for each treatment with a suitable pipette equipped with a sterile tip. Thus, each target test area receives 50 μL per visit (i.e., morning and afternoon) for a total of 100 μL of test solution per site per day. After each application of test solution to the desired target area on the forearm of the test subject, the product is distributed across the surface of the square using a sterile inoculating loop. The pipette tip and inoculating loop are discarded after each use. After all treatments have been applied, the subject remains in place for 5 minutes while the solutions air dry.

The test subjects are sampled at each target test area on the forearm (i.e., in each square 100). To sample a target area, wet a clean, sterile swab in sterile lx phosphate buffered saline+0.1% Triton X-100, and dab off excess liquid onto side of container. Discard swab solution daily. Place the swab on the target test area and apply enough pressure to bend (but not break) the swab. Continuing to apply pressure, move the tip of the swab across the target test area in a cross-hatched pattern for 5 seconds. Rotate the swab 180° and repeat. If the sample is not to be analyzed immediately, placed the swab in a 15 ml sterile conical tube and break the swab stem such that it will fit in the tube when the tube is sealed and can be conveniently removed from the tube for analysis. A stem length of one inch may be sufficient. Seal the tube and provide suitable identification on the tube (e.g., use pre-labeled tube or place a sticker label on outside of tube). If the sample is not to be analyzed immediately, but within a few hours (e.g., 1-3 hours), place the tube on ice until it is to be analyzed. If analysis is not to occur for more than a few hours (e.g., more than 3 hours), store the tube in a freezer at −80° C. until the sample is to be analyzed. Repeat the sampling procedure with a second swab on the same site using the same method, and place the second swab in 15 ml conical tube in the same way as the first swab. Store the second swab at −80° C. The second swab may be used as a backup to the first swab or used for a community analysis later (i.e., a determination of the microbial species present in the sample by DNA analysis with, for example, QPCR).

Sample Analysis

For swab samples placed on ice from above or swab samples there were taken just prior to analysis, analysis may begin immediately. Add 5 ml of 1× phosphate buffered saline+0.1% Triton X-100 to each tube to form a test solution and vortex 10 seconds to facilitate removal of microorganisms from the swab. Additional vortexing may be done to facilitate mixing just before removing test solution for plating. Measure the bacterial counts of the sample according to the Plate Count Test method described above by plating 50 μl of test solution onto a first plate using a conventional plating technique and 50 μl of a 1:10 diluted test solution (i.e., 5 μl of test solution in 45 μl of buffer solution) onto a second plate using a conventional plating technique. Transfer 200 ul of the test solution to duplicate 96-deep well plates. Freeze the 96-well plates along with any remaining test solution at −80° C. for additional analysis, as desired (e.g., QPCR). For analysis of frozen test samples, remove the tubes containing the desired samples from the freezer and allow them to sit at room temperature for about 30 minutes or until thawed. For analysis of frozen swabs without buffer, processing will be dictated by method of analysis used.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of increasing the number of anaerobic and/or aerobic skin commensal microorganisms on skin, comprising:
topically applying a cosmetic composition to a target skin surface for a sufficient period of time to increase the quantity of at least one of an anaerobic and an aerobic skin commensal microorganism, wherein the cosmetic composition includes a dermatologically acceptable carrier and an effective amount of a skin commensal prebiotic.

2. The method of claim 1, further comprising indentifying the target skin surface as being in need of a treatment that provides a skin benefit.

3. The method of claim 2, wherein the skin benefit is selected from the group consisting of improving skin appearance, improving skin feel, increasing the thickness of one or more layers of the skin, increasing the elasticity of the skin, increasing the resiliency of the skin, increasing the firmness of the skin, reducing an oily appearance of the skin, reducing a shiny appearance of the skin, reducing a dull appearance of the skin, increasing a hydration status of the skin, increasing a moisturization status of the skin, reducing an appearance of fine lines, reducing an appearance of wrinkles, improving skin texture, improving skin smoothness, improving skin exfoliation, improving skin desquamation, plumping the skin, improving skin barrier properties, improving skin tone, reducing an appearance of redness, reducing an appearance of skin blotches, improving the brightness of the skin, improving the radiancy of the skin, improving the translucency of the skin.

4. The method of claim 1, further comprising applying the cosmetic composition to the target skin surface at least once per day.

5. The method of claim 4, wherein the cosmetic composition is applied for two or more consecutive days.

6. The method of claim 1, wherein the skin commensal prebiotic remains on the skin for at least an hour.

7. The method of claim 1, wherein the quantity of skin commensal microorganism(s) is increased in vivo by at least 10%, according to the Plate Count Test.

8. The method of claim 1, wherein the prebiotic is present at an amount of from about 0.001% to about 25%.

9. The method of claim 1, wherein the prebiotic is present at an amount sufficient to increase the bacterial ATP level of at least one skin commensal microorganism in vitro by at least 80% according the ATP Test.

10. The method of claim 1, wherein the prebiotic is present at an amount sufficient to increase the bacterial ATP level of at least two skin commensal microorganisms in vitro by at least 50% according the ATP Test.

11. The method of claim 1, wherein the prebiotic is present at an amount sufficient to increase the bacterial ATP level of at least three skin commensal microorganisms in vitro by at least 25% according the ATP Test.

12. The method of claim 1, wherein the prebiotic is selected from the group consisting of galactooligosaccharide, hydroxyisoleucine, wheat dextrin, arabinogalactan, citrus fiber, pea fiber, maltodextrin, fructooligosaccharides, inulin, inulin oligofiber, mannan hydrolysates, glucomannan hydrolysates, galactomannan, gentiooligosaccharides, isomaltooligosaccharide, kiwi derived compounds, beet pulp, and rice bran.

13. The method of claim 12, wherein the galactooligosaccharide is selected from the group consisting of disaccharide, trisaccharide, tetrasaccharide, pentasaccharide, hexasaccharide and mixtures of these.

14. The method of claim 12, wherein the galactooligosaccaride is mixture of from about 20 to about 35% w/v of the disaccharide, from about 20 to about 35% w/v of the trisaccharide, from about 15 to about 25% w/v of the tetrasaccharide, and from the about 10 to about 20% w/v of the pentasaccharide.

15. The method of claim 1, wherein the skin commensal microorganism is a species selected from the group consisting of Staphylococcus genus, Corynebacterium genus, Propionibacterium genus.

16. The method of claim 1, wherein the further comprising at least one of a skin care active, a colorant, a moisturizer, a humectant, an emollient, a film former, a viscosity modifier, a pH buffering agent, a perfume, and a sunscreen agent.

17. The method of claim 1, wherein the cosmetic composition comprises at least one of a skin care active, a colorant, a moisturizer, a humectant, an emollient, a film former, a viscosity modifier, a pH buffering agent, a perfume, and a sunscreen agent.

18. A skin care regimen for maintaining or improving the condition and/or appearance of skin, the skin care regimen comprising:
using in combination
a) a first cosmetic composition comprising a dermatologically acceptable carrier and an effective amount of a skin commensal prebiotic, wherein the first cosmetic composition is a topical composition; and
b) a second composition comprising at least one of a gastrointestinal probiotic, a gastrointestinal probiotic lysate, a gastrointestinal prebiotic, and a nutritional supplement.

19. The skin care regimen of claim 18, wherein the second composition is at least one of a topical composition and an orally ingestible composition.

20. The skin care regimen of claim 18, wherein the gastrointestinal probiotic is selected from the group consisting of Bifidobacterium genus, Lactobacillus genus, Enterococcus genus, Streptococcus genus, Staphylococcus genus, Leuconostoc mesenteroides subspecies dextranicum, Pediococcus acidilactici, Sporolactobacillus inulinus, Streptococcus salvarius subspecies thermophilus, Saccharomyces cerevisiae, Saccharomyces boulardii, Bacillus cereus, Bacillus coagulans, Bacillus licheniformis, Escherichia coli strain nissle and mixtures of these.
